(19) United States
(12) Patent Application Publication
(10) Pub. No.: US 2016/0000375 A1
(43) Pub. Date: Jan. 7, 2016

(54) ENDOSCOPY CAPSULE AND METHOD OF PRODUCING AN ENDOSCOPY CAPSULE

(71) Applicant: SIEMENS AKTIENGESELLSCHAFT, München (DE)

(72) Inventors: Stefan FÖRTSCH, Weingarts (DE); Rainer KUTH, Höchstadt (DE)

(21) Appl. No.: 14/767,146

(22) PCT Filed: Jun. 25, 2013
(86) PCT No.: PCT/EP2013/063234

§ 371 Date: Aug. 11, 2015

(30) Foreign Application Priority Data
Feb. 12, 2013 (DE) 10 2013 202 257.3

(51) Int. Cl. A61B 5/00; A61B 1/04; A61B 1/00; A61B 5/1473

(52) U.S. Cl. CPC A61B 5/6861; A61B 5/42; A61B 5/1473; A61B 1/041; A61B 1/0011; A61B 2562/162; A61B 2560/0468; A61B 2562/125

(57) ABSTRACT

An endoscopy capsule is provided for detecting a metabolic product of a bacterium located in a gastrointestinal tract, wherein the endoscopy capsule has a sensor unit with two electrodes and has a capsule shell that encloses a usable space, and wherein the electrodes are routed through the capsule shell.

### CROSS-REFERENCE TO RELATED APPLICATIONS

The present patent document is a §371 nationalization of PCT Application Ser. No. PCT/EP2013/063234, filed Jun. 25, 2013, designating the United States, which is hereby incorporated by reference, and this patent document also claims the benefit of DE 10 2013 202 257.3, filed on Feb. 12, 2013, which is also hereby incorporated by reference.

### TECHNICAL FIELD

The embodiments relate to an endoscopy capsule for detecting a metabolic product of a bacterium located in a human or animal gastrointestinal tract, and to a method for producing a corresponding endoscopy capsule.

### BACKGROUND

A possible cause of symptoms affecting the area of the upper gastrointestinal tract of a patient is an infection with Helicobacter pylori bacteria.

DE 10 2010 006 969 A1 discloses a test method by which a patient may be examined for such an infection. To this end, use is made of a gastroscope with an insertion tube, at the distal end of which a sensor is arranged that reacts sensitively to ammonia. Here, use is made of the fact that Helicobacter pylori bacteria cleave urea into carbon dioxide and ammonia by the urease enzyme and that ammonia may be only detectable in relevant amounts in the stomach of a patient in the event of an infection with Helicobacter pylori bacteria. Therefore, the presence of an increased amount of ammonia and, as a consequence, also an infection with Helicobacter pylori bacteria may be deduced in the case of a corresponding reaction of the sensor, which is positioned in the stomach of a patient.

The basic principle of the function of the sensor was presented, inter alia, in the context of the presentation “Immediate detection of Helicobacter infection with a novel electrochemical system” (Gastroenterology, volume 138, issue 5, supplement 1, pages S-114, May 2010) by Helmut Neumann, Stefan Foertsch, Michael Vieth, Jonas Mudter, Rainer Kuth and Markus F. Neurath during “DIGESTIVE DISEASE WEEK 2010”. According to this, a change in an electric variable is registered metrologically when an electrode pair comes into contact with ammonia, wherein one electrode of the electrode pair reacts chemically with the ammonia.

A further possibility for detecting the presence of the pathogen in a hollow organ of the human or animal gastrointestinal tract, in particular the stomach, is described in WO 2011/141372 A1, which for its part is based on WO 2009/127528 A1. In both documents, the examination of the gastrointestinal tract is performed using an endoscopy capsule, wherein after the capsule has been swallowed, it enters the upper gastrointestinal tract and detects a metabolic product excreted by the bacterium, if such is present, within the scope of a measurement. The result of this measurement is then sent, for example, by radio transmission, to a receiver outside the patient.

### SUMMARY AND DESCRIPTION

The scope of the present invention is defined solely by the appended claims and is not affected to any degree by the statements within this summary. The present embodiments may obviate one or more of the drawbacks or limitations in the related art.

An object of the embodiments is to make available an advantageously configured endoscopy capsule, and also a method for producing a corresponding endoscopy capsule.

The endoscopy capsule serves for detecting a metabolic product of a bacterium located in a human or animal gastrointestinal tract, in particular the Helicobacter pylori bacterium, and the endoscopy capsule includes for this purpose a sensor unit with two electrodes and has a capsule shell that encloses a usable space, wherein the electrodes are routed through the capsule shell. The endoscopy capsule is kept very simple in order to minimize the production outlay and the production costs for corresponding endoscopy capsules.

The electrodes routed through the capsule shell may be needle-shaped or pin-shaped, and the free ends of the electrodes protruding from the capsule shell are expediently rounded. In this way, on the one hand, the effectiveness of the electrodes during the operation of the sensor unit is provided and, on the other hand, the risk of injury from free ends of the electrodes protruding from the capsule shell is reduced to a negligible level.

Also of advantage is an embodiment of the endoscopy capsule in which the capsule shell has the shape of an ellipsoid of revolution, wherein the maximum extent is also expediently less than 20 mm. In certain embodiments, the cylinder shape may have a longitudinal extent of at most 20 mm and a transverse extent of at most 12 mm. Since the endoscopy capsule is swallowed into the gastrointestinal tract, a suitable configuration makes the handling of the endoscopy capsule easier for a human patient and, in the case of an animal as the patient, a corresponding endoscopy capsule may be concealed in a small quantity of food.

Moreover, the capsule shell is advantageously in multiple parts, (e.g., in two parts), wherein the parts of the capsule shell may be cohesively bonded to one another, (e.g., welded or glued to one another). In this way, the capsule shell may be produced, inter alia, in such a way that the corresponding production process act for the sensor unit presents no appreciable stress. Accordingly, any electronic components that are present may not be damaged by the production process act.

Moreover, the sensor unit expediently includes a semiconductor plate, which lies in the usable space, at least in parts at a distance from the capsule shell. The semiconductor plate may serve as a support for all the electronic components of the endoscopy capsule. The electronic components are therefore held together by the semiconductor plate to form an assembly unit, which is introduced as a fixed unit into the capsule shell during the production of the endoscopy capsule. The production outlay is additionally reduced in this way.

The sensor unit may include an ammonia-sensitive sensor, as is described, for example, in the presentation “Immediate detection of Helicobacter infection with a novel electrochemical system” (Gastroenterology, volume 138, issue 5, supplement 1, pages S-114, May 2010) by Helmut Neumann, Stefan Foertsch, Michael Vieth, Jonas Mudter, Rainer Kuth and Markus F. Neurath during “DIGESTIVE DISEASE WEEK 2010”. With the aid of a corresponding ammonia-sensitive sensor, any ammonia in the gastrointestinal tract of a patient may be detected, and it is possible to infer from this the presence of an infection with Helicobacter pylori bacteria.

Also of advantage is an embodiment of the endoscopy capsule in which the position of the center of mass relative to the center of volume is predefined in such a way that, starting from the position of the center of volume, it is offset in the direction of the electrodes. Furthermore, the specific weight of the endoscopy capsule is predefined in such a way that, as a result of these two measures, the endoscopy capsule sinks to the bottom of the stomach of a human or animal patient, specifically such that the free ends of the electrodes touch the stomach wall and, as a result of this, are in contact with the gastric mucosa. This design concept has already been described in detail in WO 2011/141372 A1, incorporated by reference herein in its entirety.

In an advantageous development, the endoscopy capsule is designed exclusively to detect ammonia in a gastrointestinal tract and has no further functional units beyond this, (e.g., no optics). All that is needed for such detection is an ammonia-sensitive sensor with two electrodes and a simple voltage-measuring or current-measuring unit, a simple evaluation unit for evaluating a measurement performed with the two electrodes and the simple voltage-measuring or current-measuring unit, a radio unit for transmitting the measurement result to a receiver unit outside the patient, and an energy supply unit, (e.g., a battery), for supplying power to the electronic components present. In this way, the endoscopy capsule is kept particularly simple, wherein a set composed of endoscopy capsule and receiver unit, which is designed to display the measurement result, may be designed as a disposable article, such that a set of this kind may be offered for sale in pharmacies or drugstores as a kind of rapid test, for example. As an alternative to equipping the endoscopy capsule with a radio unit, the latter may also be equipped with a transponder. The measurement result is then transmitted according to the radio frequency identification (RFID) principle.

The capsule shell of the endoscopy capsule is also expediently made from an inert material, wherein a thermoplastic material in particular is used, (e.g., a polycarbonate material). For particularly simple production, the capsule shell or the parts of the capsule shell is or are additionally designed as an injection molded part or as injection molded parts.

The method serves for producing an endoscopy capsule, and in particular an endoscopy capsule in one of the embodiment variants described above, wherein a sensor unit with two electrodes is inserted into a part of a capsule shell, and wherein thereafter all the parts of the capsule shell are joined together in such a way that the capsule shell encloses the sensor unit in a manner impermeable to liquid, wherein the free ends of the electrodes are routed outward through the capsule shell.

The capsule shell may be designed as an ellipsoid of revolution and, in certain embodiments, may be in two parts, wherein the two parts, (e.g., the two half-shells), are produced by injection molding from a thermoplastic. With a corresponding injection molding method, the parts of the capsule shell may be produced particularly easily and cost-effectively.

According to a variant of the method, the free ends of the electrodes are driven through the capsule shell, for which purpose the capsule shell may be produced from a thermoplastic of particularly high ductility, such that the capsule shell does not splinter as the free ends of the electrodes are being driven through, and instead the capsule shell is initially only deformed, until the free ends have finally fully penetrated the material and extend outward. On account of the high ductility, the material of the capsule shell bears on the free ends of the electrodes when these are driven through, such that the capsule shell is still impermeable to liquid in the area where the free ends of the electrodes pass through the capsule shell. In this case, therefore, additional sealing of the capsule shell in the area where the electrodes pass through or outward is unnecessary, since the material of the capsule shell has a self-sealing action in this process act. Alternatively, after the electrodes have been driven through the capsule shell, the endoscopy capsule is sealed off in the area where the electrodes pass through it, for example, by melting the capsule shell in this area.

It is also of advantage if the free ends of the electrodes are heated before being driven through the capsule shell. In this case, the material of the capsule shell is as it were locally melted by the electrodes, and this likewise has a seal-sealing effect.

Particularly if provision is made to drive the free ends of the electrodes through the capsule shell, it is advantageous if the electrodes are designed with a needle shape and, in particular, if the free ends are provided with a shape tapering to a point. To provide that the electrodes protruding from the capsule shell do not cause an increased risk of injury, the free ends of the electrodes are ground cylindrically after being driven through the capsule shell.

It is expedient that the parts of the capsule shell are cohesively bonded to one another, in particular by welding or gluing.

### BRIEF DESCRIPTION OF THE DRAWINGS

FIG. 1 depicts an example of an endoscopy capsule in a sectional view.

FIG. 2 depicts the endoscopy capsule in a second sectional view in which it has been turned through 90°.

FIG. 3 depicts a block diagram of a method for producing an endoscopy capsule.

Parts corresponding to one another are provided with the same reference signs in all the figures.

### DETAILED DESCRIPTION

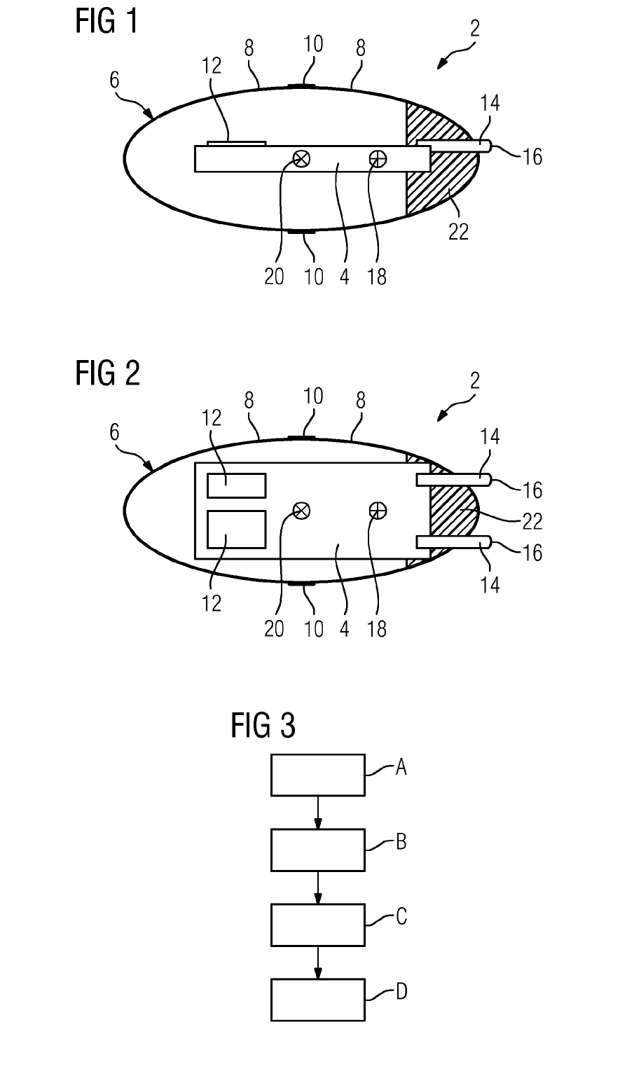

An endoscopy capsule 2 described below by way of an example is depicted in FIG. 1 in a first sectional view and is depicted in FIG. 2 in a second sectional view in which the endoscopy capsule 2 has been turned through 90° relative to the first sectional view. The endoscopy capsule 2 here includes a semiconductor plate 4, which lies in a capsule shell 6.

The capsule shell 6 has the shape of an ellipsoid of revolution and, moreover, is designed in two parts, wherein the two half-shells 8 of the capsule shell 6 are welded to each other in the area of a weld seam 10.

The semiconductor plate 4 is equipped with electronic components 12 and with two electrodes 14, wherein the two electrodes 14 are routed through the capsule shell 6 made of polycarbonate, such that their free ends 16 protrude from the capsule shell 6.

To fix the semiconductor plate 4 in the usable space enclosed by the capsule shell 6, to seal off the capsule shell 6 in the area where the electrodes 14 pass through the capsule shell 6, and to predefine the position of the center of mass 18 in such a way that the position of the center of mass 18, starting from the position of the center of volume 20, is offset in the direction of the electrodes 14, the usable space is partially filled with a synthetic resin 22 in the area of the electrodes 14.

The two electrodes 14 are stainless steel wires, of which the free ends 16 have been provided with a different coating. One of the two electrodes 14 serves as reference electrode and is coated with gold or platinum, while the other electrode 14 functions as an ammonia-sensitive electrode 14 and is coated with a layer of silver chloride and, below the latter, a layer of silver. When both electrodes 14 lie with their free ends 16 in an electrolyte, such as the stomach contents of a patient, the two electrodes 14 form, together with the electrolyte, a kind of galvanic cell, which is initially inactive on account of the silver chloride coating on one of the two electrodes 14. The silver chloride layer is insoluble in water and insoluble in stomach acid and prevents an ion flow, so that no potential difference may build up between the two electrodes 14.

In an infection with Helicobacter pylori bacteria, however, there is a relevant concentration of ammonia in the stomach contents, which reacts chemically with silver chloride, wherein a water-soluble complex is formed, such that the layer of silver chloride is removed. As soon as the layer of silver lying below is exposed, the galvanic cell is activated and a metrologically detectable potential difference builds up between the electrodes 14. The metrological detection takes place with the aid of one of the electronic components 12. A further electronic component 12 serves for radio transmission of the metrologically detected potential difference to a receiver unit situated outside the patient and on which, for example, a red or green light-emitting diode lights up, depending on whether a potential difference has or has not been detected by measurement.

If an elevated concentration of ammonia is now present in the stomach contents, the endoscopy capsule 2 registers this, and this information is sent to the receiver unit on which the green light-emitting diode then lights up, thereby signaling the presence of an infection with Helicobacter pylori bacteria.

To power the electronic components 12, a small energy supply unit, (e.g., a battery), is also located on the semiconductor plate 4.

Moreover, to be able in particular to produce a corresponding endoscopy capsule 2 cost-effectively, the method for producing endoscopy capsules 2 of this kind is kept particularly simple. The two half-shells 8 of the capsule shell 6 are produced by injection molding, and the assembly unit including the semiconductor plate 4, the electrodes 14 and the electronic components 12 is produced in the context of a prefabrication.

During the final assembly, which involves the process acts depicted in the form of a block diagram in FIG. 3, the endoscopy capsule 2 is finished. To do this, the electrodes 14 are all heated in process act A. In process act B, the semiconductor plate 4 is placed in one of the two half-shells 8 of the capsule shell 6, and the free ends 16 of the electrodes 14 are driven through the capsule shell 6 of the corresponding half-shell 8. In process act C, synthetic resin 22 is introduced into the half-shell 8, as a result of which the area in which the electrodes 14 pass through the capsule shell 6 is sealed off and, in addition, the semiconductor plate 4 is fixed in the half-shell 8. In process act D, the two half-shells 8 of the capsule shell 6 are welded to each other, such that the semiconductor plate 4 along with the electronic components 12 is enclosed in the capsule shell 6 in a manner impermeable to liquid.

According to an alternative variant of the method, process act D is followed by a refining act in which the free ends 16 of the electrodes 14 are ground cylindrically and in which the weld seam 10 is ground smooth.

The invention is not restricted to the illustrative embodiment described above. Rather, other variants of the invention may also be derived from it by a person skilled in the art, without departing from the subject matter of the invention. In particular, all of the individual features described in combination with the illustrative embodiment may also be combined with one another in another way, without departing from the subject matter of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. An endoscopy capsule for detecting a metabolic product of a bacterium located in a gastrointestinal tract, the endoscopy capsule comprising:
a sensor unit with two electrodes; and
a capsule shell that encloses a usable space,
wherein the electrodes are routed through the capsule shell.

2. The endoscopy capsule as claimed in claim 1, wherein the electrodes are needle-shaped.

3. The endoscopy capsule as claimed in claim 1, the wherein free ends of the electrodes protruding out from the capsule shell are rounded.

4. The endoscopy capsule as claimed in claim 1, wherein the capsule shell is in multiple parts cohesively bonded to one another.

5. The endoscopy capsule as claimed in claim 1, wherein the sensor unit comprises a semiconductor plate, which lies in the usable space, at least in parts at a distance from the capsule shell.

6. The endoscopy capsule as claimed in claim 1, wherein the sensor unit comprises an ammonia-sensitive sensor.

7. The endoscopy capsule as claimed in claim 1, wherein a position of a center of mass relative to a center of volume is predefined in such a way that, starting from the position of the center of volume, the center of mass is offset in a direction of the electrodes.

8. The endoscopy capsule as claimed in claim 1, wherein the endoscopy capsule is configured exclusively to detect ammonia in a gastrointestinal tract and has no functional units beyond this.

9. The endoscopy capsule as claimed in claim 1, wherein the capsule shell is made from a thermoplastic material.

10. The endoscopy capsule as claimed in claim 1, wherein the capsule shell is an injected molded part.

11. A method for producing an endoscopy capsule, the method comprising:
inserting a sensor unit with two electrodes into a part of a capsule shell such that all parts of the capsule shell are joined together in such a way that the capsule shell encloses the sensor unit in a manner impermeable to liquid, wherein free ends of the electrodes are routed outward through the capsule shell.

12. The method as claimed in claim 11, wherein the free ends of the electrodes are driven through the capsule shell.

13. The method as claimed in claim 12, further comprising:
heating the free ends of the electrodes before being driven through the capsule shell.

14. The method as claimed in claim 11, further comprising:
grinding cylindrically the free ends of the electrodes.

15. The method as claimed in claim 11, wherein the parts of the capsule shell are cohesively bonded to one another.

16. The endoscopy capsule as claimed in claim 2, wherein free ends of the electrodes protruding out from the capsule shell are rounded.

17. The endoscopy capsule as claimed in claim 16, wherein the capsule shell is in multiple parts cohesively bonded to one another.

18. The endoscopy capsule as claimed in claim 17, wherein the parts of the capsule shell are injection molded parts.

19. The endoscopy capsule as claimed in claim 4, wherein the parts of the capsule shell are injection molded parts.

20. The endoscopy capsule as claimed in claim 5, wherein the sensor unit further comprises an ammonia-sensitive sensor.
